(19) United States
(12) Patent Application Publication
(10) Pub. No.: US 2026/0021272 A1
(43) Pub. Date: Jan. 22, 2026

(54) MEDICAL TOOLS WITH MODIFIED EXTERNAL SURFACES

(71) Applicant: Boston Scientific Scimed, Inc., Maple Grove, MN (US)

(72) Inventors: Rosangel Aimee Ramos Espinoza, Boston, MA (US); Andrew James Schaubhut, Bolton, MA (US); Scott Edward Corbeil, Litchfield, NH (US); Scott E. Brechbiel, Johnston, RI (US)

(21) Appl. No.: 19/264,522

(22) Filed: Jul. 9, 2025

Related U.S. Application Data
(60) Provisional application No. 63/671,908, filed on Jul. 16, 2024.

(51) Int. Cl. A61M 25/00

(52) U.S. Cl. CPC A61M 25/0045; A61M 2025/0046

(57) ABSTRACT

According to an aspect, a medical device includes a member. The member is formed of a base material and has an exterior surface. At least a portion of the member is configured to be disposed within a body of a patient. At least a portion of the exterior surface of the member has a modification of the base material.

### CROSS-REFERENCE TO RELATED APPLICATION

This application claims priority to U.S. Provisional Patent Application No. 63/671,908, filed on Jul. 16, 2024, entitled “MEDICAL TOOLS WITH MODIFIED EXTERNAL SURFACES”, the disclosure of which is incorporated by reference herein in its entirety.

### TECHNICAL FIELD

This disclosure relates generally to bodily implants and more specifically to medical devices or tools that include elongate members, such as sphincterotomes, retrieval baskets, snares, and guidewires, that have modified external surfaces.

### BACKGROUND

Medical devices or medical tools are often inserted within a body of a patient for a medical procedure or during a delivery of a medical device. It would be useful to provide a medical device or medical tool, that has a modified external surface such that the medical device or medial tool may be water or oil repellent, non-stick, UV stable, chemically inert, lubricious, and/or durable.

### SUMMARY

According to an aspect, a medical device includes a member. The member is formed of a base material and has an exterior surface. At least a portion of the member is configured to be disposed within a body of a patient. At least a portion of the exterior surface of the member has a modification of the base material.

In some implementations, the modification of the base material is configured to provide lubriciousness or insulating properties to the member.

In some implementations, the modification of the base material includes an etching of the base material. In some implementations, the modification of the base material includes nanoscale manipulation of the base material. In some implementations, the base material being a first material, wherein the modification of the base material includes a deposition of a second material different than the first material. In some implementations, the base material being a first material, wherein the modification of the base material includes a deposition of a second material, the second material being a ceramic material. In some implementations, the modification of the base material includes an oxidation of the base material.

In some implementations, the base material is a first material, wherein the modification of the base material includes a coating of a second material different than the first material, the second material including a polymer. In some implementations, the base material is a first material, wherein the modification of the base material includes a coating of a second material different than the first material, the second material including parylene. In some implementations, the base material is a first material, wherein the modification of the base material includes a coating of a second material different than the first material, the second material including at least one of Polyolefin, polyethylene, polyurethane, polyether block amide (PEBA), a polymer with embedded ceramic, and a polymer with an embedded radiopaque material.

In some implementations, the member is an elongate member. In some implementations, the member is a guidewire. In some implementations, the member is a wire and the base material is a metal. In some implementations, the base material is a metal material. In some implementations, the modification of the base material forms a nanopattern.

According to another aspect, a medical device includes a member formed of a base material and having an exterior surface, the base material being of a first material; and a coating disposed on at least a portion of the exterior surface, the coating being formed of a second material different than the first material.

In some implementations, the first material is a metal. In some implementations, the second material includes at least one of Polyolefin, polyethylene, polyurethane, polyether block amide (PEBA), a polymer with embedded ceramic, and a polymer with an embedded radiopaque material. In some implementations, the first material is a metal and the second material includes at least one of polyethylene, polyurethane, polyether block amide (PEBA), a polymer with embedded ceramic, and a polymer with an embedded radiopaque material.

In some implementations, the member is an elongate member that is configured to at least partially be disposed within a body of a patient.

### BRIEF DESCRIPTION OF THE DRAWINGS

FIG. 1 schematically illustrates a medical device according to an implementation.

FIG. 2 is a perspective view of a medical device according to an implementation.

FIGS. 3A, 3B, 4A, 4B, 4C, 5, 6A, and 6B illustrate various nanoscale manipulations according to an implementation.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to medical devices or members that are configured to be at least partially disposed within a body of a patient. For example, in some implementations, the medical device or member may be an elongate member, such as a wire or a guidewire. In other implementations, the medical device or member may have a different shape, a different use, or a different function. The term patient may hereafter be used for a person who benefits from the medical device or the methods disclosed in the present disclosure. For example, the patient can be a person whose body receives the medical device or member, for example, in some implementations, the patient may be a human male, a human female, or any other mammal.

The implementations discussed herein may improve the performance of a medical device or other member configured to be at least partially disposed within a body of a patient. For example, in some implementations, the medical devices may have improved lubriciousness, have insulating properties, be water or oil repellent, be non-stick, be ultra-violet (UV) stable, be chemically inert, and/or be stable.

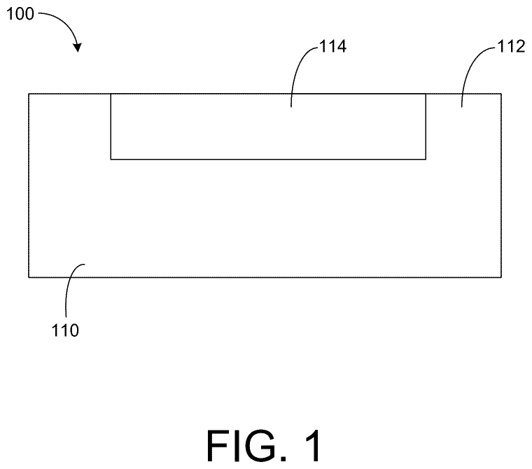

FIG. 1 schematically illustrates a medical device 100 according to an aspect. The medical device 100 includes a member 110 that has an exterior or outer surface 112. The outer surface 112 of the member includes a modification 114. In some implementations, the modification 114 provides the medical device 100 or the member 110 with beneficial characteristics or properties. For example, in some implementations, the modification 114 is configured to help the medical device 100 or member 110 have improved lubriciousness, have insulating properties, be water or oil repellent, be non-stick, be ultra-violet (UV) stable, be chemically inert, and/or be stable. In other implementations, the modification 114 helps provide other beneficial properties to the medical device 100 or the member 110.

In some implementations, the entire outer surface 112 of the member 110 includes the modification 114. In other implementations, only a portion of the outer surface 112 includes the modification 114.

In some implementations, the member 110 is formed of a base material or a first material and the modification includes a second material different than the first material. For example, in some implementations, the member 110 may be formed of a metal material and the outer surface 112 of the member may have a coating or otherwise have a different material added to the outer surface 112. For example, in some implementations, the outer surface 112 of the member 110 may be coated with a parylene material or a material that includes polymers that have a harder durometer. In other implementations, the outer surface 112 may be coated with a polyethylene material, a polyether block amide (PEBA) material, a polyurethan material, a polymeric material that has ceramic embedded therein, or a polymeric material that has radiopaque materials (such as BiOCl, bismuth, or subcarbonate) embedded therein.

In some implementations, the member 110 is formed of a base material and the modification 114 of the outer surface 112 includes a chemical modification of the base material. For example, in some implementations, the outer surface 112 is oxidized (such as through an oxidative passivation process). Oxidation of a metal surface may reduce the coefficient of friction by creating a nano or micron deep ceramic like layer at the top of the surface to help provide a more lubricous medical device 100 or member 110. In other implementations, the modification 114 of the outer surface 112 may include a plasma gas treatment. Such treatment may provide a lower surface tension and coefficient of friction of the outer surface of the member 110. In yet other implementations, the modification 114 includes an electrospinning process. Such an electrospinning process may be used to strongly bond a polymer material to the outer surface 112 of the member 110. In some implementations, the boding of the polymer material to a metallic base material would allow the coating/metal connection to transmit torque movements from one end of the medical device 100 or member 110 to an opposite end of the medical device 100 or member 110.

In some implementations, the member 110 is formed of a base material and the modification 114 of the outer surface 112 includes a material surface modification. For example, in some implementations, the modification 114 can include an etching. For example, the modification 114 can include a polymer coating etched onto the outer surface 112 of the member 110. Such coating can provide lubricating or insulative properties or both. In some implementations, the modification 114 includes a nanoscale manipulation. For example, nanoscale patterns may be formed on the outer surface 112 of the member 110. In yet other implementations, the modification 114 may include a nanofiber ceramic deposition. Such ceramic depositions may provide flexibility, porosity (which can help with lubricity), and strength to the medical device 100 or the member 110. Such ceramic depositions may be applied to the outer surface 112 via an electrospinning process, chemical vapor deposition, magnetron sputtering, solution blowing, or a laser spinning process.

The member 110 may be of any size or shape. In some implementations, the member 110 is configured to be inserted into a body of a patient. For example, in some implementations, the member 110 is configured to be entirely inserted into a body of a patient and in other implementations, only a portion of the member 110 is configured to be inserted into the body of the patient. In some implementations, the member 110 is an elongate member or a tubular shaped member. In other implementations, the member 110 has a different shape. In some implementations, the member 110 is a wire, a guidewire, a sphincterotome, a retrieval basket, a polypectomy snare, or another type of device.

The member 110 is formed of a base material. The base material may be any type of material. In some implementations, the base material is a metal material. In other implementations, the base material is nitinol. In yet other implementations, the base material is a different material, such as a non-metal material.

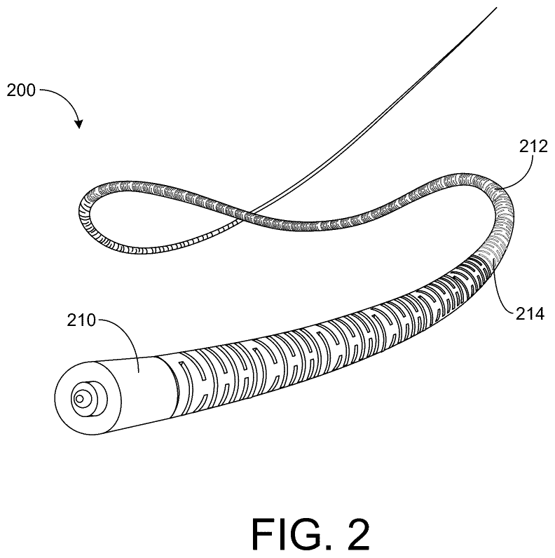

FIG. 2 illustrates a medical device 200 according to an aspect. The medical device 200 includes a member 210 that has an exterior or outer surface 212. The outer surface 212 of the member includes a modification 214. In some implementations, the modification 214 provides the medical device 200 or the member 210 with beneficial characteristics or properties. For example, in some implementations, the modification 214 is configured help the medical device 200 or member 210 have improved lubriciousness, have insulating properties, be water or oil repellent, be non-stick, be ultra-violet (UV) stable, be chemically inert, and/or be stable. In other implementations, the modification 214 helps provide other beneficial properties to the medical device 200 or the member 210.

In the illustrated implementation, the medical device 200 or the member 210 is a guidewire. In other implementations, the member 210 has a different shape. The member 210 is configured to be inserted into a body of a patient. For example, a portion of the member 210 is configured to be inserted into the body of the patient.

The member 210 is formed of a base material. The base material may be any type of material. In some implementations, the base material is a metal material. In other implementations, the base material is nitinol. In yet other implementations, the base material is a different material, such as a non-metal material.

In some implementations, the entire outer surface 212 of the member 210 includes the modification 214. In other implementations, only a portion of the outer surface 212 includes the modification 214.

In some implementations, the member 210 is formed of a base material or a first material and the modification includes a second material different than the first material. For example, in some implementations, the member 210 may be formed of a metal material and the outer surface 212 of the member may have a coating or otherwise have a different material added to the outer surface 212. For example, in some implementations, the outer surface 212 of the member 210 may be coated with a parylene material or a material that includes polymers that have a harder durometer. In other implementations, the outer surface 212 may be coated with a Polyolefin, polyethylene material, a polyether block amide (PEBA) material, a polyurethan material, a polymeric material that has ceramic embedded therein, or a polymeric material that has radiopaque materials (such as BiOCl, bismuth, or subcarbonate) embedded therein.

In some implementations, the member 210 is formed of a base material and the modification 214 of the outer surface 212 includes a chemical modification of the base material. For example, in some implementations, the outer surface 212 is oxidized (such as through an oxidative passivation process). Oxidation of a metal surface may reduce the coefficient of friction by creating a non or micron deep ceramic like layer at the top of the surface to help provide a more lubricous medical device 200 or member 210. In other implementations, the modification 214 of the outer surface 212 may include a plasma gas treatment. Such a treatment may provide a lower surface tension and coefficient of friction of the outer surface of the member 210. In yet other implementations, the modification 214 includes an electrospinning process. Such an electrospinning process may be used to strongly bond a polymer material to the outer surface 212 of the member 210. In some implementations, the boding of the polymer material to a metallic base material would allow the coating/metal connection to transmit torque movements from one end of the medical device 200 or member 210 to an opposite end of the medical device 200 or member 210.

In some implementations, the member 210 is formed of a base material and the modification 214 of the outer surface 212 includes a material surface modification. For example, in some implementations, the modification 214 can include an etching. For example, the modification 214 can include a polymer coating etched onto the outer surface 212 of the member 210. Such coating can provide lubricating or insulative properties or both.

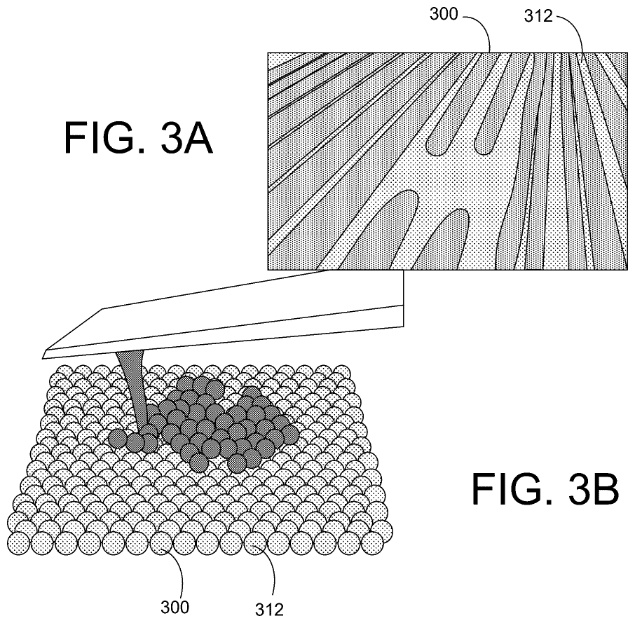

In some implementations, the modification 214 includes a nanoscale manipulation. For example, nanoscale patterns may be formed on the outer surface 212 of the member 210. As best illustrated in FIGS. 3A, 3B, 4A, 4B, 4C, 5, 6A, and 6B, a variety of different sized nanoscale patterns or nanopatterns may be used. The patterns range in size from 1 nm to larger than 100 um.

FIGS. 3A and 3B illustrate a surface 312 of a medical device 300 modified with single-asperity atomic friction. The surface 312 may be modified, for example, by using an atomic force microscope to probe and manipulate the surface of the device 300.

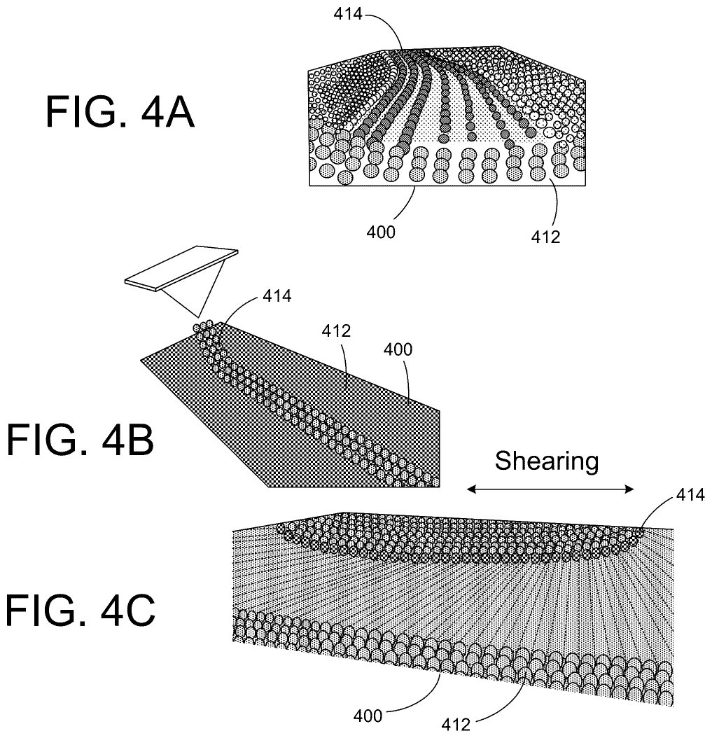

FIGS. 4A, 4B, and 4C illustrate a surface 412 of a medical device 400 having physisorbed graphic-like nano-objects 414 and physisorbed islands 416. The nano-objects may be of any shape.

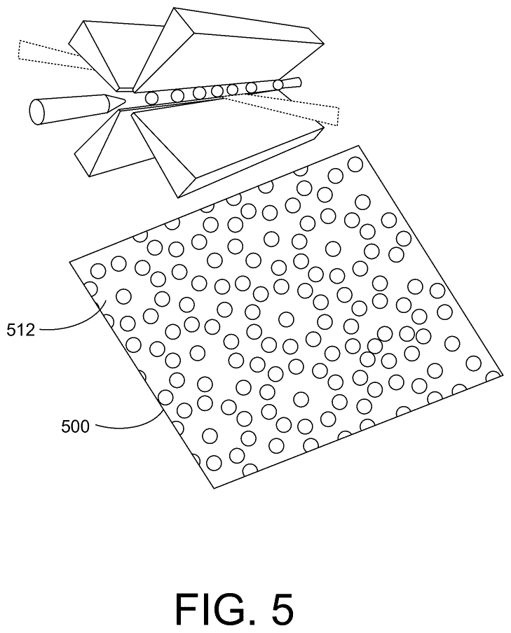

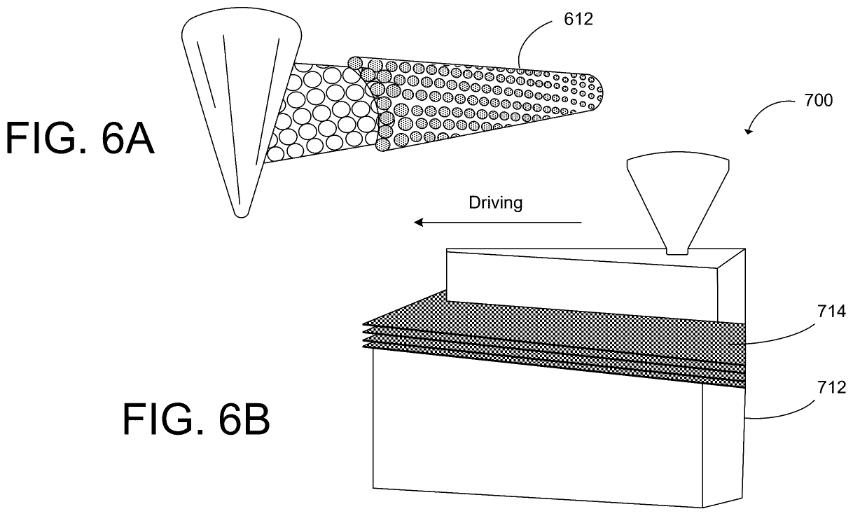

FIG. 5 illustrates a surface 512 of a medical device 500 having ion traps and colloidal suspensions. FIG. 6A illustrates long carbon nanotubes having a surface 612 with a pattern of atoms. FIG. 6B illustrates a surface 712 of a medical device 700 having two dimensional layered extended mesas 714.

In yet other implementations, the modification 214 may include a nanofiber ceramic deposition. Such ceramic depositions may provide flexibility, porosity (which can help with lubricity), and strength to the medical device 200 or the member 210. Such ceramic depositions may be applied to the outer surface 212 via an electrospinning process, chemical vapor deposition, magnetron sputtering, solution blowing, or a laser spinning process.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. A medical device, comprising:
a member formed of a base material and having an exterior surface, at least a portion of the member being configured to be disposed within a body of a patient, at least a portion of the exterior surface of the member having a modification of the base material.

2. The medical device of claim 1, wherein the modification of the base material is configured to provide lubriciousness or insulating properties to the member.

3. The medical device of claim 1, wherein the modification of the base material includes an etching of the base material.

4. The medical device of claim 1, wherein the modification of the base material includes nanoscale manipulation of the base material.

5. The medical device of claim 1, the base material being a first material, wherein the modification of the base material includes a deposition of a second material different than the first material.

6. The medical device of claim 1, the base material being a first material, wherein the modification of the base material includes a deposition of a second material, the second material being a ceramic material.

7. The medical device of claim 1, wherein the modification of the base material includes an oxidation of the base material.

8. The medical device of claim 1, the base material being a first material, wherein the modification of the base material includes a coating of a second material different than the first material, the second material including a polymer.

9. The medical device of claim 1, the base material being a first material, wherein the modification of the base material includes a coating of a second material different than the first material, the second material including parylene.

10. The medical device of claim 1, the base material being a first material, wherein the modification of the base material includes a coating of a second material different than the first material, the second material including at least one of polyethylene, polyurethane, polyether block amide (PEBA), a polymer with embedded ceramic, and a polymer with an embedded radiopaque material.

11. The medical device of claim 1, wherein the member is an elongate member.

12. The medical device of claim 1, wherein the member is a guidewire.

13. The medical device of claim 1, wherein the member is a wire and the base material is a metal.

14. The medical device of claim 1, wherein the base material is a metal material.

15. The medical device of claim 1, wherein the modification of the base material forms a nanopattern.

16. A medical device, comprising:
a member formed of a base material and having an exterior surface, the base material being of a first material; and
a coating disposed on at least a portion of the exterior surface, the coating being formed of a second material different than the first material.

17. The medical device of claim 16, wherein the first material is a metal.

18. The medical device of claim 16, wherein the second material includes at least one of polyethylene, polyurethane, polyether block amide (PEBA), a polymer with embedded ceramic, and a polymer with an embedded radiopaque material.

19. The medical device of claim 16, wherein the first material is a metal and the second material includes at least one of polyethylene, polyurethane, polyether block amide (PEBA), a polymer with embedded ceramic, and a polymer with an embedded radiopaque material.

20. The medical device of claim 16, wherein the member is an elongate member that is configured to at least partially be disposed within a body of a patient.
